(19) United States
(12) Patent Application Publication
(10) Pub. No.: US 2012/0245672 A1
(43) Pub. Date: Sep. 27, 2012

(54) Endovascular Grafts and Methods of Use

(75) Inventors: Samuel Arbefeuille, Hollywood, FL (US); Fletcher Christian, Doral, FL (US); Joseph A. Manguno, JR., Sunrise, FL (US); John C. Canning, Boynton Beach, FL (US)

(73) Assignee: Bolton Medical, Inc, Sunrise, FL (US)

(21) Appl. No.: 13/454,447

(22) Filed: Apr. 24, 2012

Related U.S. Application Data
(63) Continuation of application No. PCT/US2010/054241, filed on Oct. 27, 2010.
(60) Provisional application No. 61/255,339, filed on Oct. 27, 2009.

(51) Int. Cl. A61F 2/82; A61F 2/84

(52) U.S. Cl. 623/1.13; 623/1.23

(57) ABSTRACT

Endoluminal grafts include an endoluminal graft body and at least one vessel ingrowth component affixed to the endoluminal graft body. Endoluminal grafts can include luminal fabric and stent components that are longitudinally extendable from a first, collapsed configuration to a second, extended configuration. Endoluminal grafts can include at least one stent fixed with a proximal portion of a graft body that also includes a distal portion or a proximal portion of an endoluminal graft body retractable over at least a portion of the remaining graft body. Endoluminal grafts can be employed in methods of delivering stent grafts in patients at an aneurysm site.

### RELATED APPLICATION(S)

This application is a continuation of and claims priority to International Application No. PCT/US2010/054241, which designated the United States and was filed on Oct. 27, 2010, published in English, and claims the benefit of U.S. Provisional Application No. 61/255,339, filed Oct. 27, 2009. The entire teachings of the above application are incorporated herein by reference.

### BACKGROUND OF THE INVENTION

Minimally-invasive techniques that employ stent grafts and delivery systems for the stent grafts have been used to repair aneurysms in blood vessels, including the ascending, thoracic and abdominal aorta. The implanted stent graft generally is intended to restore blood flow through the vessel by affixing graft material across sections of the vessel weakened by the aneurysms. Currently available stent graft and delivery systems include use of stent graft systems that employ multiple components that generally have limited capacity to be adjusted in length following implantation in the blood vessel, and subsequent to vessel remodeling. In addition, currently available stent grafts have limited ability to incorporate into the blood vessel in which they are implanted. Therefore, a need exists to develop new and improved stent grafts and methods for delivering stent grafts to aneurysm sites and vessels.

### SUMMARY OF THE INVENTION

The invention is generally directed to endoluminal stent grafts and methods of using endoluminal stent grafts.

In one embodiment, the invention is an endoluminal graft that includes an endoluminal graft body and at least one vessel ingrowth component that is affixed to the endoluminal graft body.

In another embodiment, the invention is an endoluminal graft that includes a luminal fabric component and a stent component fixed to the luminal fabric component, wherein the luminal fabric component and, optionally, the stent component, is longitudinally extendable from a first, collapsed configuration, to a second, extended configuration.

In yet another embodiment, the invention is an endoluminal graft that includes an endoluminal graft body and at least one stent fixed to a proximal portion of the graft body, and wherein a distal portion of the endoluminal graft body is retractable over at least a portion of the remainder of the graft body.

In still another embodiment, the invention is a method of implanting an endoluminal graft in a patient that includes the step of delivering a stent graft that includes a luminal fabric component and a stent graft component fixed to a luminal component, wherein the luminal fabric component and, optionally, the stent component, is longitudinally extendable from a first, collapsed configuration, to a second, extended configuration, to an aneurysm site of a patient. The stent graft is radially constrained by a permeable sheath. The permeable sheath is partially retracted from the stent graft to thereby partially deliver a proximal end of the stent graft to a point proximal to the aneurysm site. The partially deployed stent graft is longitudinally adjusted by moving the fabric and stent components of the stent graft from the first collapsed configuration to the second extended configuration to thereby substantially span a longitudinal length of the aneurysm site, and the permeable sheath is retracted from the patient to thereby implant the endoluminal graft in the patient.

In a further embodiment, the invention is a method of implanting an endoluminal graft including the step of delivering an endoluminal graft, that includes an endoluminal graft body and at least one vessel ingrowth component that is affixed to at least one end of the endoluminal graft body, to an aneurysm site of the patient. The endoluminal graft can, optionally, be radially constrained by a permeable sheath. The endoluminal graft is retracted to thereby deliver the endoluminal graft to the aneurysm site.

In a further embodiment, the invention is a method for implanting an endoluminal graft in a blood vessel of a patient including the steps of deploying an endoluminal graft in a retracted position in a blood vessel, wherein a proximal end of the graft is retracted over a portion of the remainder of the graft and wherein the proximal end is fixed to the blood vessel. The distal end of the graft is drawn in a direction distal to the patient, thereby reducing the portion of the graft that is retracted over the remainder of the graft, until the graft is implanted in the vessel.

In still another embodiment, the invention is a method of implanting an endoluminal graft in a blood vessel of a patient, comprising the steps of delivering an endoluminal graft in a retracted position in a blood vessel, wherein a proximal end of the graft is retracted over a portion of the remainder of the graft and wherein the proximal end is fixed to the vessel and a distal end of the endoluminal graft is fixed to a clasp or a tether of a delivery device; drawing the distal end of the endoluminal graft in a direction distal to the patient, thereby reducing the portion of the graft that is retracted over the remainder of the graft; and releasing the distal end of the endoluminal graft from the clasp or the tether of the delivery device to thereby position the endoluminal graft in the blood vessel.

The grafts and methods of the invention have several advantages, including securing proximal and distal ends of the graft in the blood vessel at the site of implantation in a minimally traumatic manner that permits in-situ adjustment in the length of the stent graft by the physician and decrease the potential for graft migration during vessel remodeling. Further, ingrowth of tissue into at least one vessel ingrowth component of the endoluminal graft provides for greater long-term securing and sealing of the implanted endoluminal graft.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A depicts an embodiment of an endoluminal graft that includes an endoluminal graft body and a vessel ingrowth component of the invention.

FIG. 1B depicts an embodiment of a vessel ingrowth component of an endoluminal graft of the invention.

FIG. 2 depicts another embodiment of an endoluminal graft of the invention that includes an endoluminal graft body and a vessel ingrowth component.

FIGS. 3A and 3B depict embodiments of endoluminal grafts of the invention that include a vessel ingrowth component fixed at the proximal end and the distal end of the endoluminal graft body.

FIG. 4 depicts an embodiment of the invention of a bifurcated endoluminal graft that includes main body and bifurcation components.

FIGS. 5A and 5B depict embodiments of endoluminal grafts of the invention with a luminal fabric component and stent component fixed to the luminal fabric component that longitudinally expands from a first, collapsed configuration (FIG. 5A) to a second, extended configuration (FIG. 5B).

FIGS. 6A and 6B depict embodiments of endoluminal grafts of the invention with a luminal fabric component and stent components fixed to the luminal fabric component that longitudinally expands from a first, collapsed configuration (FIG. 6A) to a second, extended configuration (FIG. 6B).

FIGS. 7A and 7B depict embodiments of endoluminal grafts of the invention with a luminal fabric component and stent component fixed to the luminal fabric component that longitudinally expands from a first, collapsed configuration (FIG. 7A) to a second, extended configuration (FIG. 7B).

FIGS. 8A, 8B and 8C depict embodiments of endoluminal grafts of the invention with a luminal fabric component and stent component fixed to the luminal fabric component that longitudinally expands from a first, collapsed configuration (FIGS. 8A and 8C) to a second, extended configuration (FIG. 8B).

FIGS. 9A and 9B depict embodiments of an endoluminal graft of the invention with a bifurcated luminal fabric component and a stent component fixed to the bifurcated luminal fabric component that longitudinally expand from a first, collapsed configuration (FIG. 9A) to a second extended configuration (FIG. 9B).

FIGS. 10A, 10B, 10C and 10D depict embodiments of endoluminal grafts of the invention that include an endoluminal graft body and a distal (FIG. 10A) or a proximal (FIGS. 10B and 10C) portion of the endoluminal graft body that is retractable over at least a portion of the remainder of the graft body.

FIGS. 10E-10G depict an embodiment of an anchor having at least one hook and a stent at a proximal portion of an endoluminal graft of the invention.

FIGS. 11A-11D depict an embodiment of a method of the invention, wherein an endoluminal graft is implanted in an aortic arch of a patient.

FIGS. 12A-12C depict an embodiment of a method of the invention, wherein an endoluminal graft is implanted in an abdominal aorta of a patient.

FIG. 13 depicts an embodiment of a distal end of an endoluminal graft of the invention that includes a clasp at a distal end of the endoluminal graft.

### DETAILED DESCRIPTION OF THE INVENTION

The features and other details of the invention, either as steps of the invention or as combinations as parts of the invention, will now be particularly described and pointed out in the claims. It will be understood that the particular embodiments of the invention are shown by way of illustration and not as limitations of the invention. The principle features of this invention can be employed in various embodiments without departing from the scope of the invention. A description of example embodiments of the invention follows.

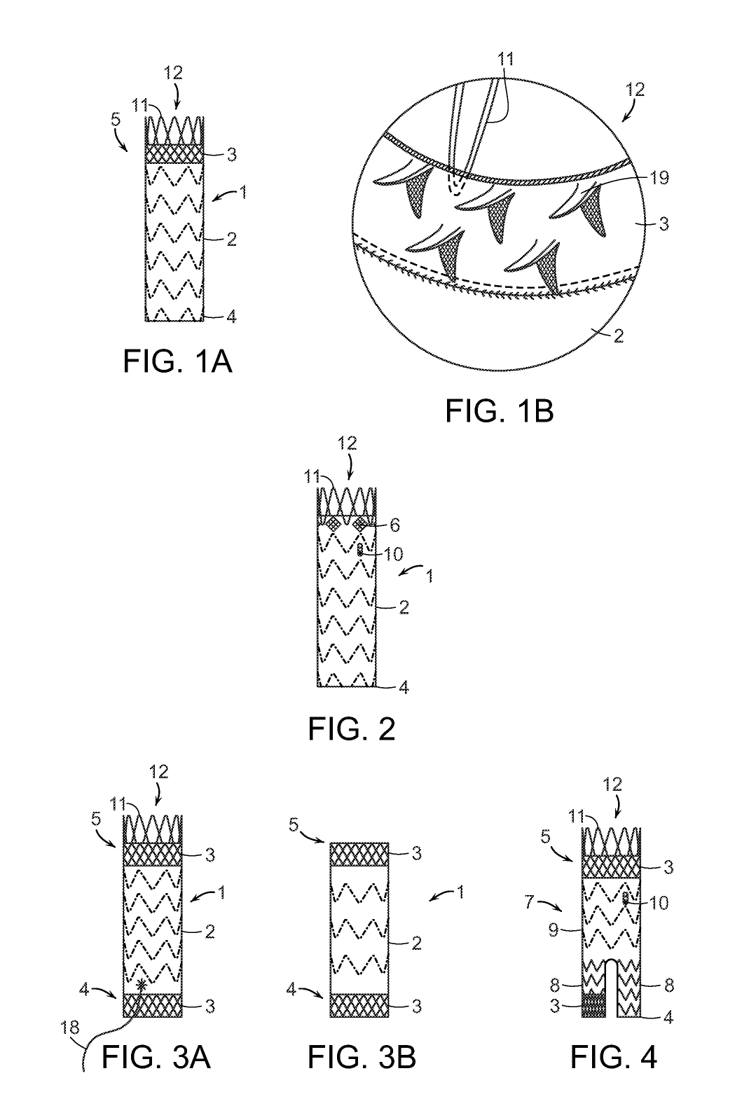

As shown in exemplary FIGS. 1-3, in the invention is endoluminal graft 1 that includes endoluminal graft body 2 and at least one vessel ingrowth component 3, 6 that is a fixed to the endoluminal body. Vessel ingrowth component 3, 6 can be fixed to an external surface of the endoluminal graft body. Vessel ingrowth component 3, 6 can cover or not cover the endoluminal graft body, specifically the fabric of the graft body. The vessel ingrowth component has an advantage of replacing, in part, the graft fabric to secure and stabilize the endoluminal graft in the vessel wall. Vessel ingrowth component 3, 6 employed in the endoluminal grafts described herein can be fixed to at least one end (proximal end 5, distal end 4) of the endoluminal graft body. For example, endoluminal graft 1 can include at least two vessel ingrowth components, wherein one vessel ingrowth component 3, 6 is affixed to distal portion 4 of the endoluminal graft body and second vessel ingrowth component 3, 6 is affixed to proximal portion 5 of endoluminal graft body 2. Vessel ingrowth component 3, 6 can be affixed to endoluminal graft body 2 by, for example, sewing or gluing vessel ingrowth component 3, 6 to endoluminal graft body 2. Vessel ingrowth component 3, 6 can be arranged in a pattern, such as diamond-shaped pattern 6 as shown in FIG. 2, or as continuous sleeve 3, as shown in FIGS. 1A, 3A, 3B and 4 on the surface of endoluminal graft body 2.

The vessel ingrowth component can be formed of at least one member selected from the group consisting of stainless steel, titanium, nickel titanium, cobalt and chromium cobalt. The vessel ingrowth component can be made by well-established techniques, such as at least one technique selected from the group consisting of laser cutting, stamping, heat shape setting nitinol and braiding techniques. For example, a vessel ingrowth component that includes stainless steel, titanium, nickel titanium, cobalt and chromium cobalt can be formed by laser cutting the stainless steel, titanium, nickel titanium, cobalt or chromium cobalt. In one embodiment, the vessel ingrowth implant is collapsible, such as by folding. In a specific embodiment, the vessel ingrowth implant is self-expanding, such as by virtue of being formed, at least in part, of nitinol.

As shown in FIG. 4, in an embodiment, the endoluminal graft that includes the vessel ingrowth component can be bifurcated endoluminal graft 7 that includes at least one bifurcation component 8 at one end of bifurcated endoluminal graft 7 and main graft body 9. Bifurcated endoluminal graft 7 can include at least one vessel ingrowth component 3 affixed to distal portion 4 of at least one bifurcated component 8 of the bifurcated endoluminal graft, which can, optionally, be used in combination with at least one additional vessel ingrowth component 3 affixed to main graft body 9 of bifurcated endoluminal graft 7, as shown in FIG. 4. Main graft body 9 of bifurcated endoluminal graft 7 can also include at least one vessel ingrowth component 3 affixed to main graft body 9, for example, at proximal end 5 of the bifurcated endoluminal graft 7.

An endoluminal graft that includes an endoluminal graft body and at least one vessel ingrowth component that is affixed to the endoluminal graft body can further include at least one fixation component 12, such as bare stent 11 (FIG. 4) affixed to at least one end of the endoluminal graft body that fixes the prosthesis to a blood vessel wall of a patient. In an embodiment, fixation component 12 is affixed to distal portion 4 of endoluminal graft body 2 or main graft body 9 of bifurcated endoluminal graft 7. In another embodiment, fixation component 12 is affixed to proximal portion 5 of endoluminal graft body 2 or proximal portion 5 of bifurcated endoluminal graft 7. In a further embodiment, fixation components of the endoluminal graft body of the invention can be affixed to distal and proximal portions of the endoluminal graft body.

As shown in FIG. 1B, vessel ingrowth component 3, 6 can include at least one tab 19. Vessel ingrowth component 3, 6 can be formed, for example, of stainless steel, titanium, nickel titanium, cobalt and chromium cobalt from which vessel ingrowth component 3, 6 can be laser cut. Vessel ingrowth component 3 (FIG. 1B) can include tab 19 that protrudes between about 0.5 mm to 3 mm in length (e.g., about 0.5 mm, about 1 mm, about 1.5 mm, about 2 mm, about 2.5 mm, about 3 mm).

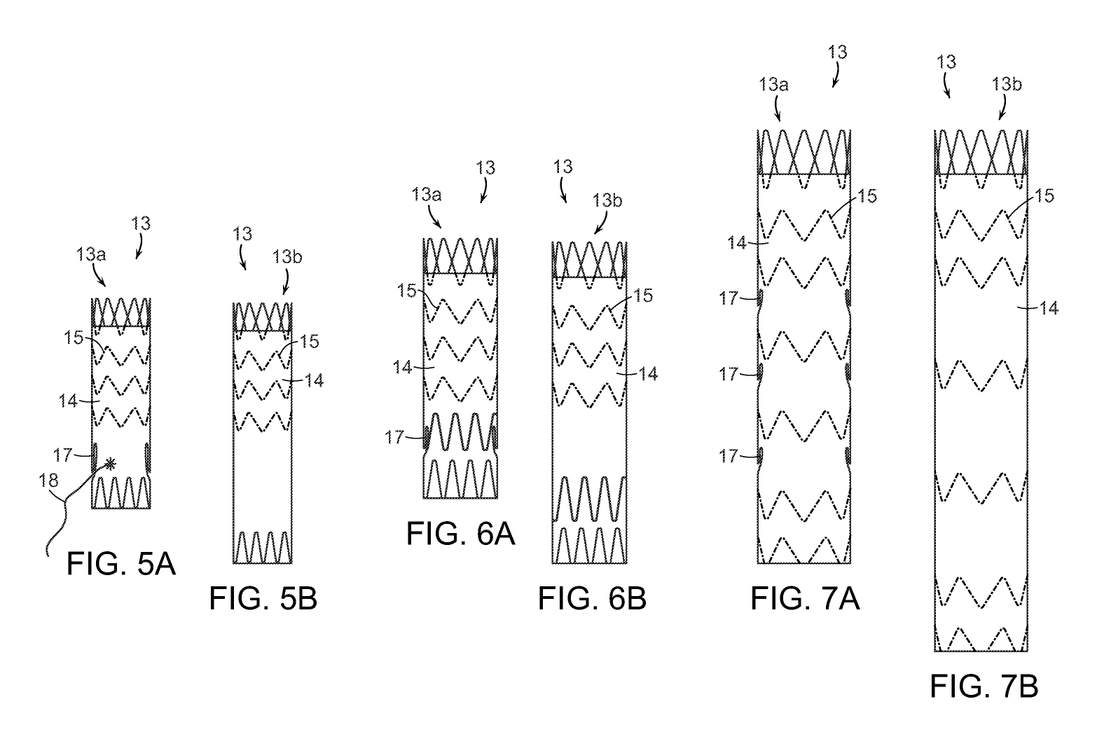

As shown in FIGS. 5A, 5B, 6A, 6B, 7A, 7B, 8A, 8B and 8C, in other embodiments, the invention is endoluminal graft 13, comprising luminal fabric component 14 and stent component 15 fixed to the luminal fabric component, wherein the luminal fabric component, and, optionally, stent component (not shown), is longitudinally extendable from first, collapsed configuration 13a to second, extended configuration 13b.

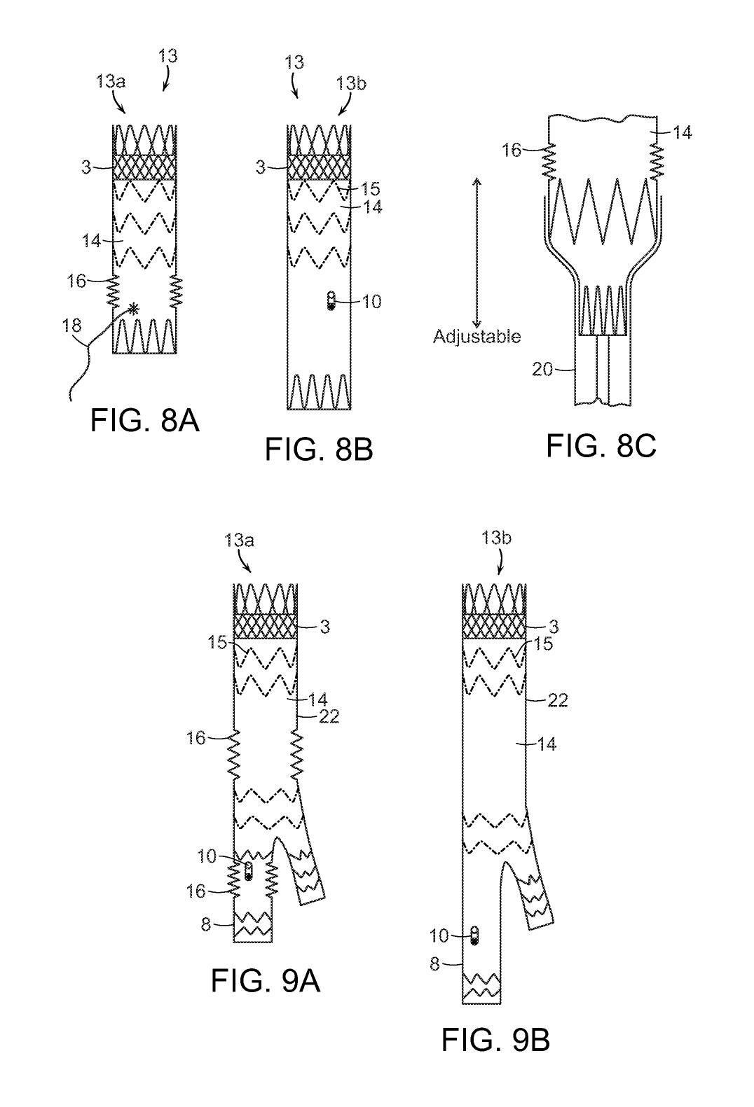

Stent component 15 can be fixed, such as by sewing, to at least one member selected from the group consisting of the inside of the luminal fabric component and the outside of the luminal fabric component. In one embodiment, luminal fabric component 14 includes at least one corrugation 16 (FIGS. 8A, 8C, 9A). In another embodiment, the luminal fabric component includes at least one fold 17 (FIGS. 5A, 6A, 7A, 8A and 9A). In an embodiment, the luminal fabric component includes one fold or corrugation, two folds or corrugations, three folds or corrugations or 4 folds or corrugations (e.g., FIGS. 5A and 7A). Luminal fabric components with corrugations can be purchased as prefabricated corrugated luminal fabric components (Atex Technologies, Pinebluff, N.C. and Vascutek Ltd, Renfrewshire, Scotland, UK). Corrugations or folds in the luminal fabric components can be made during the heat-set phase of construction of the luminal fabric component on a suitable mandrel. For example, corrugations or folds in the luminal fabric component can be made by compressing the luminal fabric component along a longitudinal axis of the mandrel.

The folds or corrugations can be between about 5 millimeters to about 40 millimeters in length (e.g., about 5 millimeters, about 10 millimeters, about 15 millimeters, about 20 millimeters, about 25 millimeters, about 30 millimeters, about 35 millimeters and about 40 millimeters).

Corrugations can be alternating ridges or grooves. Folds can be smooth overlaps of material.

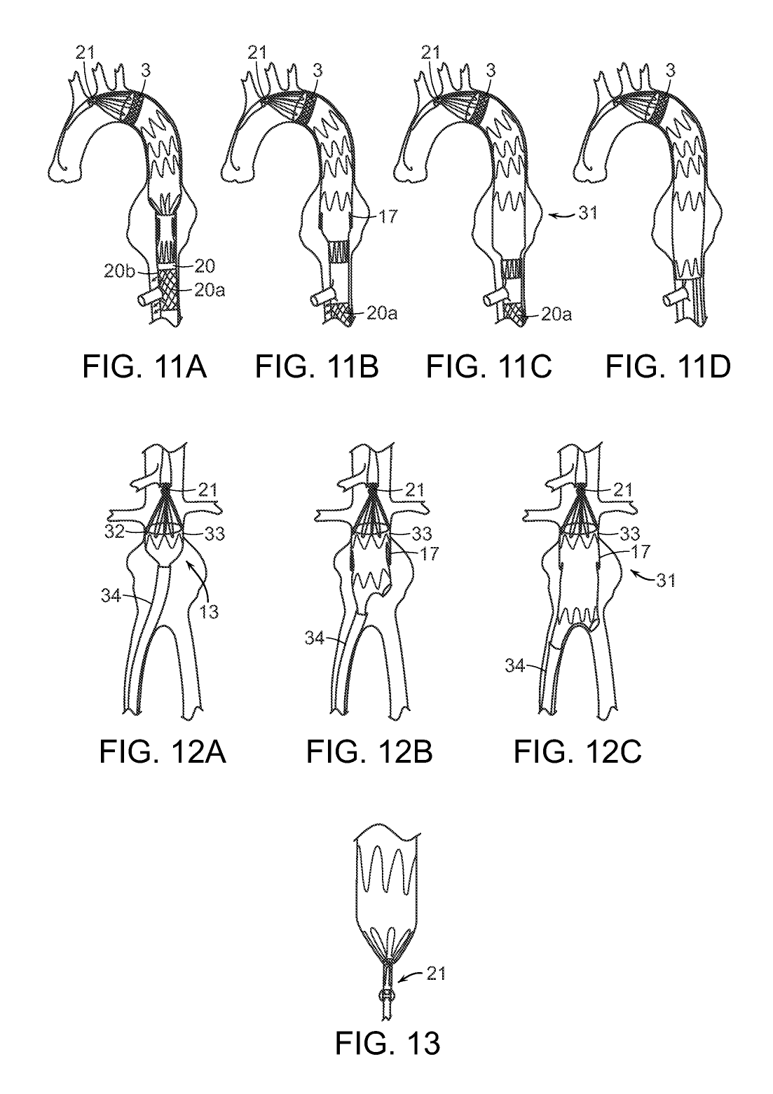

The endoluminal graft that includes a luminal fabric component and a stent component fixed to the luminal fabric component, wherein the luminal fabric component, and, optionally, the stent component, is longitudinally extendable from first, collapsed configuration 13a to second, extended configuration 13b. The endoluminal graft can further include tether 18 (FIGS. 3A, 8A, 10B, 10C and 10D) or clasp 21 (FIGS. 11A-11D and 12A-12C) affixed to an end of the endoluminal graft, such as a proximal end or a distal end of the endoluminal graft, whereby the endoluminal graft can be remotely extended from the first to the second configuration by retraction of the tether or the clasp. Remote extension of the endoluminal graft from a first to a second configuration can be achieved by retraction of a component of a delivery device from the patient in which the endoluminal graft is implanted. The tether or clasp component may be secured to the endoluminal graft by a hook means, or other suitable removable means. The tether can be removable from the graft. The tether can be formed of suture material. The clasp can be formed of at least one member selected from the group consisting of stainless steel, titanium, nickel titanium, cobalt and chromium cobalt. The clasp can be a clasp as described in, for example, U.S. application Ser. Nos. 11/353,927 and 11/449,337.

The endoluminal graft that is longitudinally extendable from a first, collapsed configuration to a second, extended configuration can further include at least one vessel ingrowth component 3 and at least one radiopaque marker 10 (FIGS. 8B, 9A and 9B).

In one embodiment, the endoluminal graft that includes a luminal fabric component and stent component fixed to the luminal fabric component, wherein the luminal fabric component, and, optionally, the stent component, is longitudinally extendable from a first, retracted configuration to second, extended configuration can be bifurcated luminal fabric component 22 (FIGS. 9A and 9B). The bifurcated luminal fabric component can include at least one fold 17 (FIGS. 5A, 6A and 7A), at least one corrugation 16 in the main graft body or at least one bifurcation component 8 (FIG. 9A). The endoluminal grafts described herein can further include permeable sheath 20 about the endoluminal graft and, optionally, a permeable sheath that is removable from the graft (FIG. 8C). Permeable sheath 20 can be formed of at least one member selected from the group consisting of polyester, braided mesh, nitinol and stainless steel. In a particular embodiment, permeable sheath 20 includes at least one through-hole 20a, as depicted in FIGS. 11A-11D. Through-hole 20a permits perfusion or continued flow of blood during deployment of an endoluminal graft. The through-holes can be defined by a mesh or fabric of perforation portion of the permeable sheath 20a or as distinct openings, as shown in FIGS. 11A-11D. Typically, the through-holes have a diameter equal to or greater than about 0.6 mm². The through-holes permit relatively continuous blood flow through the endoluminal graft into the vessel in the direction shown by arrows 20b during implantation of the endoluminal graft.

In another embodiment, the endoluminal grafts can include standard sheath 34 (also referred to as “introducer sheath”) (FIGS. 12A, 12B and 12C). Standard sheath 34 can be formed of at least one member selected from the group consisting of polymer, such as PTFE, a high density polymer and PVAC. Standard sheath 34 can be used to introduce a device to deliver endoluminal grafts of the invention through vascular access, such as the femoral artery.

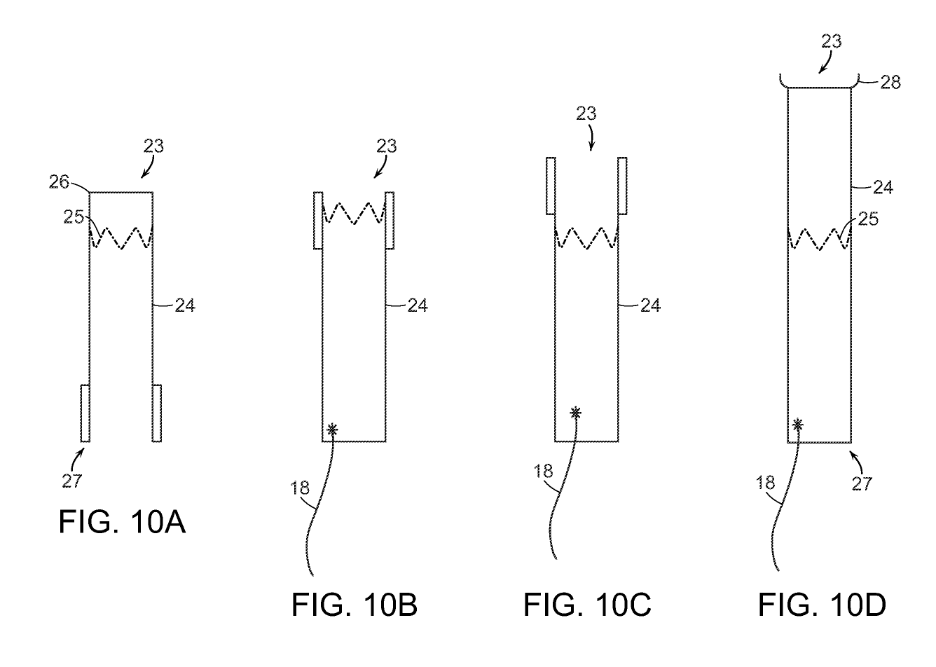

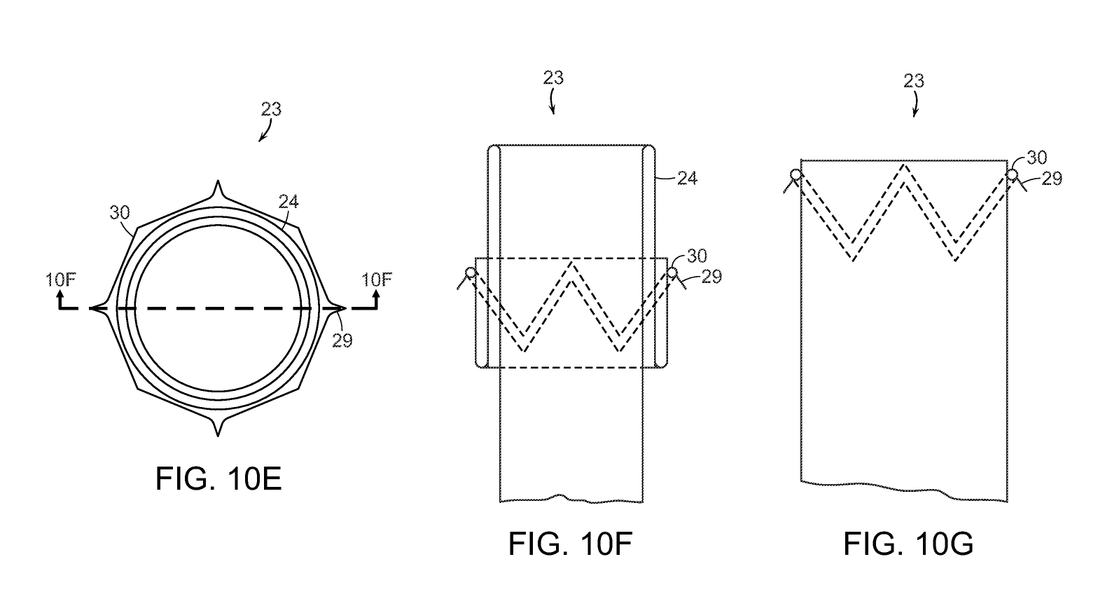

As shown in FIGS. 10A-10D, in still another embodiment, the invention is endoluminal graft 23 that includes endoluminal graft body 24 and at least one stent 25 fixed to proximal portion 26 of the endoluminal graft body, and wherein distal portion 27 of endoluminal graft body 24 is retractable over at least a portion of the remainder of endoluminal graft body 24 (FIG. 10A or, alternatively, FIG. 10B). Endoluminal graft 23 can further include anchor 28 at proximal portion 26 to secure the graft in a blood vessel while endoluminal graft 23 is in a retracted position.

In another embodiment, proximal portion 26 of endoluminal graft 23 is retractable over at least a portion of the remainder of the endoluminal graft 23 (FIGS. 10B-10D). In a further embodiment, distal portion 27 of endoluminal graft 23 is retractable over at least a portion of the remainder of endoluminal graft 23 (FIG. 10A).

Another embodiment, shown in FIGS. 10E through 10G includes proximal stent 30 at a proximal end of endoluminal graft 23. In an embodiment, anchor 28 includes at least one of hook 29 and stent 30. Endoluminal graft body 24 is doubly-folded at a proximal end of the endoluminal graft 23 to preserve an external position of stent 30, shown in FIG. 10E, during movement in a direction directed to the patient from a retracted portion shown in FIG. 10F to an extended position, shown in FIG. 10G.

Stents employed in the endoluminal grafts of the invention can be self-expanding stents. Endoluminal grafts can include proximal bare stent 11 (see FIGS. 1A, 2, 3A and 4) or can include a non-bare stent (FIG. 3B).

The endoluminal grafts of the invention can further include at least one radiopaque marker 10 affixed to the graft material or the stent of the endoluminal graft. Radiopaque markers can be located at the proximal end of endovascular graft, at the distal end of endovascular graft or at both the proximal end and distal end of the endoluminal graft. In a bifurcated endoluminal graft, a shorter bifurcation component can include lateral radiopaque markers, at least one of which is at the distal opening of the shorter bifurcation component.

The endoluminal graft that includes an endoluminal graft body and at least one stent fixed to a proximal portion of the graft body, wherein the distal portion of the endoluminal graft body retractable over at least a portion of the remainder of the graft body, and can further include a tether or a clasp extending from the distal portion of the graft body.

As shown in FIGS. 11A-11D and 12A-12C, in still another embodiment, the invention is a method of implanting an endoluminal graft in a patient. The method includes delivering a stent graft, that includes an luminal fabric component and a stent component fixed to the luminal fabric component, wherein the luminal fabric component and, optionally, the stent component, is longitudinally extendable from a first, collapsed configuration, to a second, extended configuration, to aneurysm site 31 of the patient. The stent graft is radially constrained by permeable sheath 20 or standard sheath 34, as shown in FIGS. 11A-11D and 12A-12C. The permeable sheath or the standard sheath is partially retracted (FIGS. 11A, 11B and 12A-12B) from the stent graft to thereby partially deliver proximal end 32 of the stent graft to point proximal 33 to the aneurysm site. The aneurysm site can be a thoracic aneurysm site, as shown in FIGS. 11A-11D, or an abdominal aorta aneurysm site as shown in FIGS. 12A-12C. The partially-delivered stent graft is longitudinally adjusted by moving the fabric and stent components of the stent graft from a first collapsed configuration (FIG. 12A) to a second extended configuration (FIG. 12C) to thereby span a longitudinal length of the aneurysm. The endoluminal graft is implanted in the patient by retracting permeable sheath 20 or standard sheath 34 from the patient. The method has the advantage of permitting adjustments to the length of the graft inside blood vessels near and at an aneurysm site, which can vary in length and width, depending on the patient and associated trauma. In one embodiment, at least one of the proximal end of the endoluminal graft and the distal end of the endoluminal graft can include anchor 28 to further secure the endoluminal graft to the inner wall of the vessel in which the endoluminal graft is implanted.

In another embodiment, the endoluminal graft further includes tether 18 (FIGS. 3A, 5A, 8A, 10B and 10D) or clasp 21 (FIGS. 11A-11D and 12A-12C) at one end (proximal or distal) of the endoluminal graft. The tether or clasp permits the endoluminal graft to be remotely extended from a first to a second configuration by pulling the tether or clasp from a location remote to the aneurysm site. For example, remote extension can be accomplished by movement of the tether by the surgeon or movement of components of a delivery device to thereby move the clasp. The endoluminal graft can be remotely extended from a first to a second configuration by a tether after a proximal portion of the prosthesis has been delivered to a point of implantation at the aneurysm site. The proximal end of the endoluminal graft can further include an anchor that assists in securing the proximal end of the endoluminal graft while pulling on the tether to extend the endoluminal graft from a first to a second configuration.

In yet another embodiment, the invention is a method of implanting an endoluminal graft, comprising the steps of delivering an endoluminal graft that includes an endoluminal graft body and at least one vessel ingrowth component 3 (as shown in FIGS. 11A-11D) that is affixed to at least one end of the endoluminal graft to an aneurysm site of the patient, the endoluminal graft being radially constrained by permeable sheath 20 or standard sheath 34. As shown in FIGS. 11A-11D, permeable sheath 20 is retracted from the endoluminal graft to thereby deliver the endoluminal graft to the aneurysm site after which the permeable sheath is refracted from the patient to thereby implant the endoluminal graft. As shown in FIGS. 12A-12C standard sheath 34 is retracted from the endoluminal graft to thereby deliver the endoluminal graft to the aneurysm site after which the standard sheath is refracted from the patient to thereby implant the endoluminal graft.

In yet another embodiment, the invention is a method for implanting an endoluminal graft in a blood vessel of a patient comprising deploying an endoluminal graft in a retracted position in the blood vessel, wherein a proximal end of the graft is retracted over a portion of the remainder of the graft and wherein the proximal end is affixed to the blood vessel. A distal end of the graft is drawn in a direction distal to the patient to thereby reduce the portion of the graft that is retracted over the remainder of the graft until the graft is implanted in the blood vessel.

In an additional embodiment, the invention is a method of implanting an endoluminal graft in a blood vessel of a patient by delivering an endoluminal graft in a retracted position in a blood vessel, wherein a proximal end of the graft is retracted over a portion of the remainder of the graft and wherein the proximal end is fixed to the vessel and a distal end of the endoluminal graft is fixed to clasp 21 or tether 18 of a delivery device. The distal end of the endoluminal graft is drawn in a direction distal to the patient, thereby reducing the portion of the graft that is retracted over the remainder of the graft. The distal end of the endoluminal graft is released from the clasp or the tether of the delivery device to thereby position the endoluminal graft in the blood vessel.

The endoluminal graft and methods described herein permit more accurate placement of distal or proximal ends of prosthesis and fixation of the endoluminal graft to the intima of the blood vessel in an area adjacent to the aneurysm site.

Systems, stent grafts and delivery devices and components of systems, stent grafts and delivery devices as described in U.S. Pat. No. 7,763,063 and U.S. application Ser. No. 10/784,462, filed on Feb. 23, 2004; Ser. No. 11/348,176, filed on Feb. 6, 2006; Ser. No. 11/353,927, filed on Feb. 13, 2006; Ser. No. 11/449,337, filed on Jun. 8, 2006; Ser. No. 11/699,700, filed on Jan. 30, 2007; Ser. No. 11/699,701, filed on Jan. 30, 2007; Ser. No. 11/700,609, filed on Jan. 30, 2007; Ser. No. 11/700,510, filed on Jan. 31, 2007; Ser. No. 11/701,867, filed on Feb. 1, 2007; Ser. No. 11/828,675, filed on Jul. 26, 2007; Ser. No. 11/828,653, filed on Jul. 26, 2007; Ser. No. 12/137,592, filed on Jun. 12, 2008; Ser. No. 11/701,876, filed on Feb. 1, 2007; 61/164,545, filed on Mar. 30, 2009; Ser. No. 12/459,387, filed on Jun. 30, 2009, and Ser. No. 12/623,431, filed Mar. 12, 2010, the teachings of all of which are hereby incorporated by reference in their entirety, can be employed in the systems, stent grafts and delivery devices described herein.

While this invention has been particularly shown and described with references to example embodiments thereof, it will be understood by those skilled in the art that various changes in form and details may be made therein without departing from the scope of the invention encompassed by the appended claims.

What is claimed is:

1. An endoluminal graft, comprising:
a) an endoluminal graft body; and
b) at least one vessel ingrowth component that is affixed to the endoluminal graft body.

2. The endoluminal graft of claim 1, wherein the vessel ingrowth component is affixed to at least one end of the endoluminal graft body.

3. The endoluminal graft of claim 2, wherein one vessel ingrowth component is affixed to a distal portion of the endoluminal graft body and a second vessel ingrowth component is affixed to a proximal portion of the endoluminal graft body.

4. The endoluminal graft of claim 1, wherein the vessel ingrowth component is formed of at least one member selected from the group consisting of stainless steel, titanium, nickel titanium, cobalt and chromium cobalt.

5. The endoluminal graft of claim 1, wherein the endoluminal graft body is a bifurcated endoluminal graft body that includes at least one bifurcation component at an end of the endoluminal graft body.

6. The endoluminal graft of claim 5, wherein the vessel ingrowth component is affixed to a distal portion of at least one bifurcation component of the bifurcated graft body.

7. The endoluminal graft of claim 5, wherein the bifurcated endoluminal graft body further includes at least one radiopaque marker.

8. The endoluminal graft of claim 1, further including at least one fixation component affixed to at least one end of the endoluminal graft body that fixes the prosthesis to a blood vessel wall of a patient.

9. The endoluminal graft of claim 8, wherein the fixation component is affixed to the distal portion of the endoluminal graft body.

10. The endoluminal graft of claim 8, wherein the fixation component is affixed to the proximal portion of the endoluminal graft body.

11. An endoluminal graft, comprising:
a) a luminal fabric component; and
b) a stent component fixed to the luminal fabric component, wherein the fabric component, and, optionally, the stent component, is longitudinally extendable from a first, collapsed configuration, to a second, extended configuration.

12. The endoluminal graft of claim 11, wherein the luminal fabric component includes at least one corrugation.

13. The endoluminal graft of claim 11, wherein the luminal fabric component includes at least one fold.

14. The endoluminal graft of claim 11, further including a tether or a clasp affixed to an end of the endoluminal graft, whereby the endoluminal graft can remotely be extended from the first to the second configuration.

15. The endoluminal graft of claim 14, wherein the tether is removable from the graft.

16. The endoluminal graft of claim 11, wherein the luminal fabric component is a bifurcated luminal fabric component.

17. The endoluminal graft of claim 11, further including a permeable sheath about the endoluminal graft.

18. The endoluminal graft of claim 11, wherein the permeable sheath is removable from the graft.

19. An endoluminal graft, comprising:
a) an endoluminal graft body; and
b) at least one stent fixed to a proximal portion of the graft body, and wherein a distal portion of the endoluminal graft body is retractable over at least a portion of the remainder of the graft body.

20. The endoluminal graft of claim 19, wherein the graft further includes an anchor at the proximal end, whereby the graft can be secured in a blood vessel while the graft is in a retracted position.

21. The endoluminal graft of claim 20, wherein the anchor includes at least one of a hook and a stent.

22. The endoluminal graft of claim 21, wherein the stent is self-expanding.

23. The endoluminal graft of claim 22, further including a tether extending from the distal portion of the graft.

24. A method of implanting an endoluminal graft in a patient, comprising the steps of:
a) delivering a stent graft that includes a luminal fabric component and a stent component fixed to the luminal fabric component, wherein at least one of the luminal fabric component, and optionally, the stent component, is longitudinally extendable from a first, collapsed configuration, to a second, extended configuration, to an aneurysm site of a patient;
b) partially retracting the stent graft to thereby partially deliver a proximal end of the stent graft to a point proximal to the aneurysm site; and
c) longitudinally adjusting the partially delivered stent graft by moving the fabric and stent components of the stent graft from the first collapsed configuration to the second extended configuration to thereby substantially span a longitudinal length of the aneurysm site, thereby implanting the endoluminal graft in the patient.

25. The method of claim 24, wherein the stent graft is radially constrained by a permeable sheath.

26. The method of claim 24, wherein the endoluminal graft further includes at least one of a tether or a clasp at one end of the endoluminal graft, whereby the endoluminal graft can remotely be extended from the first to the second configuration and pulling on the tether or the clasp from a location remote to the aneurysm to thereby extend the endoluminal graft from the first to the second position after a proximal portion of the prosthesis has been delivered to a point of implantation at the aneurysm site.

27. The method of claim 24, wherein the endoluminal graft is a bifurcated endoluminal graft.

28. The method of claim 24, wherein the stent graft further includes at least one fixation component affixed to at least one end of the stent graft.

29. A method of implanting an endoluminal graft, comprising the steps of:
a) delivering an endoluminal graft that includes an endoluminal graft body and at least one vessel ingrowth component that is affixed to at least one end of the endoluminal graft body to an aneurysm site of a patient; and
b) retracting the endoluminal graft to thereby deliver the endoluminal graft to the aneurysm site and implant the endoluminal graft

30. The method of claim 29, wherein the endoluminal graft is radially constrained by a permeable sheath.

31. A method for implanting an endoluminal graft in a blood vessel of a patient, comprising the steps of:
a) deploying an endoluminal graft in a retracted position in a blood vessel, wherein a proximal end of the graft is retracted over a portion of the remainder of the graft, and wherein the proximal end is fixed to the blood vessel; and
b) drawing a distal end of the graft in a direction distal to the patient, thereby reducing the portion of the graft that is retractive over the remainder of the graft until the graft is implanted in the blood vessel.

32. A method of implanting an endoluminal graft in a blood vessel of a patient, comprising the steps of:
a) delivering an endoluminal graft in a retracted position in a blood vessel, wherein a proximal end of the graft is retracted over a portion of the remainder of the graft and wherein the proximal end is fixed to the vessel and a distal end of the endoluminal graft is fixed to a clasp or a tether of a delivery device;
b) drawing the distal end of the endoluminal graft in a direction distal to the patient, thereby reducing the portion of the graft that is retracted over the remainder of the graft; and
c) releasing the distal end of the endoluminal graft from the clasp or the tether of the delivery device to thereby position the endoluminal graft in the blood vessel.
